SEQUENCE LISTING (6 sequences)

SEQ ID NO: 1 - DNA, 663 bp, Bacillus thuringiensis
atgaatcctaatatacttgtgataaatggtaatacgaagaatcaagaatttattcaaaatctcctgattataaagaaattcatgattgatacggtaaatgatggtatagaaggaattattcgctttcaaaagaaagtgtacgatctagttatactagatgttatgtcacctaatttagatggatttagtatatgtaaaattataagatcacaatctaaagtgccaattatcatgttatctacaataaaggacgaaagtattgagattaaaggatttcaatttggtattgatgactttattactctaccatgttcagttgagctattttattataggatagaagccattctacgaggacgaaattcaactgattcattgtcattaatacagtttcaagaaatatcactaaatcctgattcgtatatagtgtatcttaatgggcagaagaaaaagctaacgacaaaagaatttgatatgctacatatatttttaagaaatccagggaaagtattatctagagaatttttgctgaatcaagtgtggggatatgattattatggagatccacgagtcatagatgcgtatataaaaaaactacgcaaaaagttaagtattccgtatataaaaacaataacaggcgttggttataaactagacacataa

SEQ ID NO: 2 - DNA, 65 bp, Artificial Sequence
  Promoter activated by CpcR consensus sequence
  Variant (1)..(1): Replace = a
  Variant (6)..(6): Replace = t
  Variant (11)..(11): Replace = t
  Variant (12)..(12): Replace = t or g
  Variant (13)..(13): Replace = c or t
  Variant (14)..(14): Replace = a
  Variant (17)..(17): Replace = t
  Variant (18)..(18): Replace = c
  Variant (20)..(20): Replace = t or g
  Variant (25)..(25): Replace = a
  Variant (27)..(27): Replace = c
  Variant (30)..(30): Replace = c or t
  Variant (31)..(31): Replace = a or g
  Variant (32)..(32): Replace = c
  Variant (33)..(33): Replace = g or t
  Variant (34)..(34): Replace = g
  Variant (36)..(36): Replace = t
  Variant (37)..(37): Replace = c or t
  Variant (39)..(39): Replace = t
  Variant (44)..(44): Replace = c
  Variant (45)..(45): Replace = c or g
  Variant (47)..(47): Replace = a
  Variant (49)..(49): Replace = t
  Variant (52)..(52): Replace = t
  Variant (54)..(54): Replace = c
  Variant (56)..(56): Replace = t
  Variant (57)..(57): Replace = g
  Variant (58)..(58): Replace = t
  Variant (59)..(59): Replace = g or t
  Variant (61)..(61): Replace = g or t
  Variant (62)..(62): Replace = a
  Variant (64)..(64): Replace = g
  Variant (65)..(65): Replace = t
gtgaataaaaaaagcacaaaatttccatcatcagttaacatgtaattgctagattgaaaacgtac

SEQ ID NO: 3 - DNA, 65 bp, Bacillus thuringiensis
gtgaacaaaaagtacatcaaatttccatcctcggtttacatgtacttgctatattggagaggtac

SEQ ID NO: 4 - DNA, 65 bp, Bacillus thuringiensis
gtgaacaaaaaacacatcagatttccatctgagatttacatgtacttgctatattggtaaggtac

SEQ ID NO: 5 - DNA, 65 bp, Bacillus thuringiensis
gtgaataaaataagcacaatatttccctcatcagttcatatgtcattgctagactgaagatgtat

SEQ ID NO: 6 - DNA, 65 bp, Bacillus thuringiensis
atgaataaaatttgcacaagatttacatcaactataaacatgtagtagttatatttaatacatgt

(19) United States
(12) Patent Application Publication
(10) Pub. No.: US 2019/0327977 A1
(43) Pub. Date: Oct. 31, 2019

(54) USE OF THE CPCR REGULATOR GENE FOR OBTAINING NEW RECOMBINANT STRAINS OF BACILLUS THURINGIENSIS WITH REDUCED SPORULATION CAPACITY

(71) Applicants: Institute National De La Recherche Agronmique, Paris (FR); Institute of Plant Protection, Chinese Academy of Agricultural Sciences, Beijing (CN)

(72) Inventors: Didier Lereclus, Oulins (FR); Leyla Slamti, Paris (FR); Fu Ping Song, Beijing (CN); Chao Deng, Beijing (CN); Jie Zhang, Beijing (CN)

(73) Assignees: INSTITUT NATIONAL DE LA RECHERCHE AGRONOMIQUE, Paris (FR); Institute of Plant Protection, Chinese Academy of Agricultural Sciences, Beijing (CN)

(21) Appl. No.: 16/475,541

(22) PCT Filed: Jan. 5, 2018
(86) PCT No.: PCT/EP2018/050288

§ 371 Date: Jul. 2, 2019

(30) Foreign Application Priority Data
Jan. 5, 2017 (EP) 17305011.3

(51) Int. Cl. A01N 63/02; C07K 14/325; C12N 15/75; C12N 3/00; C12P 21/02; C12N 1/20

(52) U.S. Cl. CPC A01N 63/02; C07K 14/325; C12N 1/20; C12N 3/00; C12P 21/02; C12N 15/75

(57) ABSTRACT

The present disclosure relates to the use of a cpcR regulator gene, which directs the expression of promoters of genes encoding Cry proteins, for reducing the sporulation of a strain of Bacillus thuringiensis, new recombinant strains of Bacillus thuringiensis and uses thereof as biopesticide.

### FIELD OF THE INVENTION

The present invention relates to the use of a cpcR regulator gene for reducing the sporulation of a recombinant strain of Bacillus thuringiensis, a recombinant strain of Bacillus thuringiensis comprising the cpcR gene and uses thereof in particular as biopesticides.

### BACKGROUND OF THE INVENTION

Bacteria and sporulation Bacillus species are rod-shaped, endospore-forming aerobic or anaerobic, Gram-positive bacteria. The many species of the genus are able to live in every natural environment. Only one endospore is formed per cell. The spores are resistant to heat, cold, radiation, dessiccation, and disinfectants. At the onset of the sporulation, secondary metabolites are produced by Bacillus sp.. These secondary metabolites may be: enzymes, antibiotics and insecticides.

Bacillus larvae, Bacillus lentimorbus, Bacillus popilliae, Bacillus sphaericus, and Bacillus thuringiensis are pathogens of specific groups of insects (Bacillus, Medical Microbiology). Thus, some bacillus are used as the active ingredients of insecticides.

Bacillus thuringiensis (Bt) is a bacterium present in soil, which produces spores and synthetize crystals releasing proteins which are toxic to insects and classified as Cry1, -2, -3, -4, . . . , according to their peptide sequences (Crickmore et al., 1998). More particularly, Bt strains produce insecticidal crystals in the mother cell of sporulating bacteria which are used as biopesticides for controlling undesirable insects, such as Lepidoptera, Coleoptera and mosquitoes (Schnepf et al., 1998). At the end of sporulation, the crystal is found alongside the spore. The promoters of genes encoding these crystal toxins are known. They are for example the promoters controlling the expression of genes cry1, cry2, cry3, cry4 (Agaisse et al., 1995; Deng et al., 2014). The promoters of the cry1, cry2 and cry4 genes are recognized by RNA polymerases associated to the specific sigma factors, Sigma E or Sigma K; the promoters of the cry3 genes are recognized by RNA polymerase associated to the vegetative sigma factor, Sigma A.

Cry Toxins

Cry toxins have specific activities against insect species of the orders Lepidoptera, Diptera, Coleoptera, Hymenoptera and against nematodes. When insects ingest crystals, their alkaline digestive tracts denature the crystals, making them soluble and thus amenable to being cut with proteases found in the insect gut, which liberate the toxin from the crystal (Schnepf et al., 1998). The Cry toxin is then inserted into the insect gut cell membrane, paralyzing the digestive tract and forming a pore. The insect stops eating and starves to death (Schnepf et al., 1998).

Bt-Based Biopesticides

Bt is successfully used as a biopesticide for more than 50 years (Bravo et al., 2011 and Sanahuja et al., 2011). However, the efficiency of these Bt-based biopesticides is assessed to only a few days on the leaf surface. Indeed, the chemistry of the leaf surface, proteases and sunlight contribute to the degradation of Cry proteins.

Today, all the Bt-based biopesticides used in the world consist of a mixture of spores and crystals. However, toxins present in crystals are rapidly destroyed because they are released in the external medium at the end of the sporulation process. Further, the dissemination of bacterial spores may be detrimental for Human and environment.

It remains a need to provide safer Bt-based biopesticides wherein the stability of the Cry toxins is improved.

### DETAILED DESCRIPTION OF THE INVENTION

In order to solve these problems, the inventors studied a Bt strain, called LM1212 that presents the unique ability to differentiate into crystal-producers or spore-formers. Transcriptional analysis suggested that the parasporal crystal phenotype results from a new type of cell differentiation associated with a novel regulation mode of cry gene expression (Deng et al., 2015). As specified above, all biopesticides known until now consist of a mixture of spores and crystals. The characterization of the unusual LM1212 strain, in which the crystal is not produced in the mother cell of sporulating cells, but only in a subpopulation of non-sporulating cells, expanded inventor's understanding of the parasporal crystal phenotype in Bt. Thereby, the inventors have identified surprisingly and unexpectedly a unique regulatory sequence gene present in this LM1212 and called “crystal producing cells regulator” (cpcR). They showed that the introduction of this cpcR gene in a typical Bt strain drastically allows to reduce sporulation rate.

The inventors have characterized this regulator gene and its nucleotide sequence, which comprises the sequence SEQ ID NO: 1.

Thus, this CpcR regulator directs the expression of the cry gene promoters in Bacillus thuringiensis LM1212.

The inventors have also shown that the use of this regulator allows the expression of heterologous cry genes, as the cry1Ab gene encoding a lepidopteran active toxin, and the production of crystal inclusions in non sporulating Bt cells.

Moreover, the introduction of the cpcR gene in a typical Bt strain drastically reduced its sporulation rate while maintaining or increasing the expression of cry genes. The results obtained by the inventors indicate that this expression system allows the production of insecticidal Cry proteins in a Spo⁻ genetic background. Thus, the invention provides safer Bt-based biopesticides by preventing the dissemination of bacterial spores. Moreover, the production and the stability of the Cry toxins in dedicated bacterial cells are improved since the toxins are protected from the environmental degradation, such as UV irradiation.

Accordingly, the present invention relates to the use of the cpcR regulator gene of the sequence SEQ ID NO: 1 for reducing the sporulation of a recombinant strain of Bacillus thuringiensis.

The nucleotide sequence SEQ ID NO: 1 is:

ATGAATCCTAATATACTTGTGATAAATGGTAATACGAAGAATCAAGAATT
TATTCAAAATCTCCTGATTATAAAGAAATTCATGATTGATACGGTAAATG
ATGGTATAGAAGGAATTATTCGCTTTCAAAAGAAAGTGTACGATCTAGTT
ATACTAGATGTTATGTCACCTAATTTAGATGGATTTAGTATATGTAAAAT
TATAAGATCACAATCTAAAGTGCCAATTATCATGTTATCTACAATAAAGG
ACGAAAGTATTGAGATTAAAGGATTTCAATTTGGTATTGATGACTTTATT
ACTCTACCATGTTCAGTTGAGCTATTTTATTATAGGATAGAAGCCATTCT
ACGAGGACGAAATTCAACTGATTCATTGTCATTAATACAGTTTCAAGAAA
TATCACTAAATCCTGATTCGTATATAGTGTATCTTAATGGGCAGAAGAAA
AAGCTAACGACAAAAGAATTTGATATGCTACATATATTTTTAAGAAATCC
AGGGAAAGTATTATCTAGAGAATTTTTGCTGAATCAAGTGTGGGGATATG
ATTATTATGGAGATCCACGAGTCATAGATGCGTATATAAAAAAACTACGC
AAAAAGTTAAGTATTCCGTATATAAAAACAATAACAGGCGTTGGTTATAA
ACTAGACACATAA

“Recombinant strain” in the present invention means that one or more fragments of genes or one or more entire genes is/are inserted by genetic engineering, in a parental strain.

“Reducing the sporulation” in the present invention means that the rate of sporulation of the recombinant strain of Bt of the invention is reduced at least 2 fold preferably at least 5 fold, more preferably at least 10 fold, even more preferably at least 15 fold compared to the sporulation rate of the parental Bt strain.

In order to characterize the cpcR regulator gene, first the inventors determined a DNA fragment from LM1212, responsible for the activation of a promoter of a gene encoding a Cry protein in the LM1212. Then, they reduced this DNA fragment until identifying the gene responsible for this effect. After identification, the inventors validated that cpcR is responsible for a) the activation of cry gene expression in the LM1212 strain and b) the reduction of the sporulation rate. Finally, the inventors tested the use of the cpcR in a recombinant strain of Bt and obtained a reduction of the sporulation rate and an increase of the production of Cry1 Ab. These results confirm the interest to use the cpcR gene in a recombinant strain of Bt and the use of this recombinant strain of Bt as new biopesticide.

The cpcR regulator directs the expression of several promoters of genes encoding Cry and Cyt proteins, said promoters having the consensus sequence SEQ ID NO:2.

The nucleotide sequence SEQ ID NO: 2 is:

X₁TGAAX₂AAAAX₃X₄X₅X₆CAX₇X₈AX₉ATTTX₁₀CX₁₁TCX₁₂X₁₃X₁₄X₁₅X₁₆T
X₁₇X₁₈AX₁₉ATGTX₂₀X₂₁TX₂₂GX₂₃TAX₂₄AX₂₅TX₂₆X₂₇X₂₈X₂₉AX₃₀X₃₁TX₃₂
X₃₃, with:
X₁ = A or G; X₂ = C or T; X₃ = A or T; X₄ = A, T or
G; X₅ = A, C or T; X₆ = A or G; X₇ = C or T; X₈ = A
or C X₉ = A, T or G; X₁₀ = A or C; X₁₁ = A or C;
X₁₂ = A, C or T; X₁₃ = A, G or T; X₁₄ = A or C; X₁₅ =
A, G or T; X₁₆ = A or G; X₁₇ = A or T; X₁₈ = A, C or
T; X₁₉ = C or T; X₂₀ = A or C; X₂₁ = A, C or G; X₂₂ =
A or T; X₂₃ = C or T; X₂₄ = G or T; X₂₅ = C or T;
X₂₆ = G or T; X₂₇ = A or G; X₂₈ = A or T; X₂₉ = A, G
or T; X₃₀ = C, G or T; X₃₁ = A or G; X₃₂ = A or G;
X₃₃ = C or T.

Preferably, the promoter activated by CpcR, is selected from the group consisting of: P₃₂ of the sequence SEQ ID NO: 3, P₄₁ of the sequence SEQ ID NO: 4, P₃₅ of the sequence SEQ ID NO: 5, P₄₅ of the sequence SEQ ID: 6.

More preferably, the promoter activated by CpcR is P₃₅.

According to the invention, genes encoding the proteins activated by CpcR in the LM1212 strain, are preferably cry genes, more preferably cry32Val, cry41Cal, cry45Ba1, cry74Aa1, cry32Wa1 and cry35-like, even more preferably cry32Wa1 and cry35-like.

According to the invention, genes encoding proteins, in particular toxins, produced by the recombinant strain of Bacillus thuringiensis are preferably cry genes or cyt genes.

Preferably, the cry and cyt genes encoding toxins produced by the recombinant strain of the invention under the control of CpcR are:

1) cry genes encoding toxins active against crop pests, specifically lepidopteran and coleopteran insects. These cry genes belong for example to the classes cry1, cry2, cry3, cry8 or cry9.

2) cry genes encoding toxins active against insect vectors of human diseases, specifically dipteran insects as mosquitoes and simuli. These cry genes belong for example to the classes cry4 and cry11.

3) cry genes encoding toxins active against nematodes. These cry genes belong for example to the classes cry5, cry6, cry14 and cry21.

4) cyt genes encoding toxins active against insect vectors of human diseases, specifically dipteran insects as mosquitoes and simuli. These cyt genes belong for example to the classes cyt1 and cyt2.

More preferably, the genes encoding toxins produced by the recombinant strain of the invention under the control of CpcR are cry1, cry2, cry3, cry4, cry5, cry 6, cry8, cry9, cry11, cry14, cry21, cyt1 or cyt2 genes.

The activity spectrum of the Cry and Cyt toxins against insect pests is described in: Van Frankenhuizen, K., et al., 2009. The activity spectrum of the Cry toxins against nematodes is described in: Wei et al., 2003.

More preferably, the toxin produced by the recombinant strain of the invention is Cry1Ab, a lepidopteran active toxin, encoded by the cry1Ab gene.

The inventors showed that the expression of the cpcR gene allows the expression of heterologous cry genes, such as the cry1Ab gene, and the production of crystal inclusions in non-sporulating Bt cells.

The present invention also relates to a recombinant strain of Bacillus thuringiensis, which is characterized in that it comprises:
a) At least one gene encoding Cry and/or Cyt toxins,
b) At least one promoter having the sequence SEQ ID NO: 2, allowing the expression of said at least one gene encoding Cry and/or Cyt toxins, and,
c) A cpcR regulator gene of sequence SEQ ID NO: 1, which directs the expression of said at least one promoter.

The main feature of this recombinant strain, in the present invention, is its reduced sporulation rate in comparison with the parental strain.

According to the invention, this bacterium may be used living or dead. Indeed, the viability of the bacteria is not required to ensure the insecticidal or the nematicidal activity of this recombinant strain of Bacillus thuringiensis. Since CpcR negatively affects sporulation, the number of viable spores will be reduced or even abolished.

Preferably, the parental strain of Bacillus thuringiensis is the Bacillus thuringiensis kurstaki HD73 (type strain of the serotype 3a, 3b, 3c).

Preferably, the promoter present in the recombinant strain of Bacillus thuringiensis is selected from the group consisting of: P₃₂ of the sequence SEQ ID NO: 3, P₄₁ of the sequence SEQ ID NO: 4, P₃₅ of the sequence SEQ ID NO: 5, P₄₅ of the sequence SEQ ID: 6.

More preferably, the promoter present in the recombinant strain of Bacillus thuringiensis is P₃ₛ.

According to the invention, the promoter may be cloned on a plasmid, which is different to the plasmid carrying the cpcR regulator gene or the promoter may be cloned on the same plasmid as the cpcR regulator gene.

More preferably, the promoter is cloned on the same plasmid as the cpcR regulator gene.

According to the invention, genes encoding the toxins in the recombinant strain of Bacillus thuringiensis are preferably cry or cyt genes, more preferably cry1, cry2, cry3, cry4, cry5, cry6, cry8, cry9, cry11, cry 14, cry 21, cyt1 or cyt2 genes, more preferably, cry1, cry2, cry3, cry8 or cry9 genes.

Preferably, the toxins produced by the recombinant strain of the invention are Cry1 and Cry2 toxins.

More preferably, the toxin produced by the recombinant strain of the invention is Cry1Ab, a lepidopteran active toxin, encoded by the cry1Ab gene.

The present invention also relates to the use of the recombinant strain of Bacillus thuringiensis as described above as biopesticide.

“Biopesticide” in the present invention refers to pesticides derived from natural materials, such as bacteria, for example, in the present invention. This general term “biopesticide” includes the term “biocide” which is more used in a context of controlling vectors, such as vectors that transmit pathogens responsible for mammalian diseases.

In a particular embodiment, the recombinant strain of Bacillus thuringiensis is used for protecting cultures. The “biopesticide” producing CpcR-regulated Cry proteins will be used for controlling crop pests, specifically lepidopteran insects such as those belonging to the families Tortricidae, Noctuidae and Pyralidae which damage crops like cotton, cabbage, corn, soybean, grapevine and any other extensive or gardener cultures.

Preferably, the toxins produced by the recombinant strain of the invention for protecting cultures are: Cry1, Cry2, Cry3, Cry 8 and Cry9 toxins.

In another particular embodiment, the recombinant strain of Bacillus thuringiensis is used to control vectors, especially vectors that transmit pathogens responsible for mammalian diseases, preferably human diseases, such as insect vectors, in particular mosquitoes or simuli. In such a particular embodiment, the recombinant strain of Bacillus thuringiensis will harbor CpcR-regulated cry genes encoding toxins specifically active against dipteran insects.

Preferably, the toxins produced by the recombinant strain of the invention in the use to control vectors are toxins belonging, for example, to the classes Cyt1, Cry4 and Cry11.

In another particular embodiment, the recombinant strain of Bacillus thuringiensis is used to control nematodes, especially those responsible for mammalian diseases, preferably human diseases. In such a particular embodiment, the recombinant strain of Bacillus thuringiensis will harbor CpcR-regulated cry genes encoding toxins specifically active against nematodes.

Preferably, the toxins produced by the recombinant strain of the invention in the use to control nematodes are toxins belonging, for example, to the classes Cry5, Cry6, Cry14 and Cry21.

The present invention also relates to a method for obtaining a recombinant strain of Bacillus thuringiensis as described above, comprising the steps of introducing in said strain both:
1—a genetic construction comprising at least one gene encoding a toxin under the control of a promoter having the sequence SEQ ID NO: 2, and
2—an expression system comprising the CpcR regulator of the sequence SEQ ID NO: 1.

The two steps of this method can be performed in the order as defined above or in the reverse order but also simultaneously by introducing a construct carrying the three cited elements.

Preferably, the genetic construction as defined in step 1 of said method and the expression system as defined in step 2 of said method are on the same plasmid. In such an embodiment, step 1 and step 2 can be performed simultaneously.

Preferably, the parental strain of Bacillus thuringiensis is the Bacillus thuringiensis kurstaki HD73.

Preferably, the promoter having the sequence SEQ ID NO: 2 is selected from the group consisting of: P₃₂ of the sequence SEQ ID NO: 3, P₄₁ of the sequence SEQ ID NO: 4, P₃₅ of the sequence SEQ ID NO: 5, P₄₅ of the sequence SEQ ID: 6.

More preferably, said promoter is P₃₅ of sequence SEQ ID NO: 2.

Preferably, the genes encoding a toxin are cry1, cry2, cry3, cry 4, cry5, cry6, cry8, cry9, cry11, cry14, cry21 or cyt genes, more preferably cry1, cry2, cry3, cry8 or cry9 genes.

More preferably, the gene encoding a toxin is cry1Ab gene.

The promoter which directs the transcription of the cpcR gene is any promoter functional in Bt. The choice of the more appropriate promoter may depend in particular on the type of expression (i.e. constitutive or inducible) that one wishes to obtain. Promoters may be constitutive promoters, i.e. promoters which are active in cells and under most environmental conditions, or cell specific promoters which are active only or mainly in certain cell types, and inducible promoters that are activated by physical or chemical stimuli.

The genetic construction will be done on Bacillus plasmids based on the replicons pBC16, pHT315 or pHT73 and carrying a cry gene downstream from a CpcR-regulated promoter.

The genetic construct can be introduced in a strain of Bacillus thuringiensis by electroporation (Lereclus et al., 1989; Bone et al. 1989; Koehler et al. 1994; Mahillon et al., 1999; Peng et al. 2009), by using recombination procedure as previously described (Lereclus et al., 1992), or by heterogramic conjugation (Trieu-Cuot et al., 1987).

In addition to the above features described, the invention further comprises other features which will emerge from the following description, which refers to examples illustrating the present invention, as well as to the appended figures.

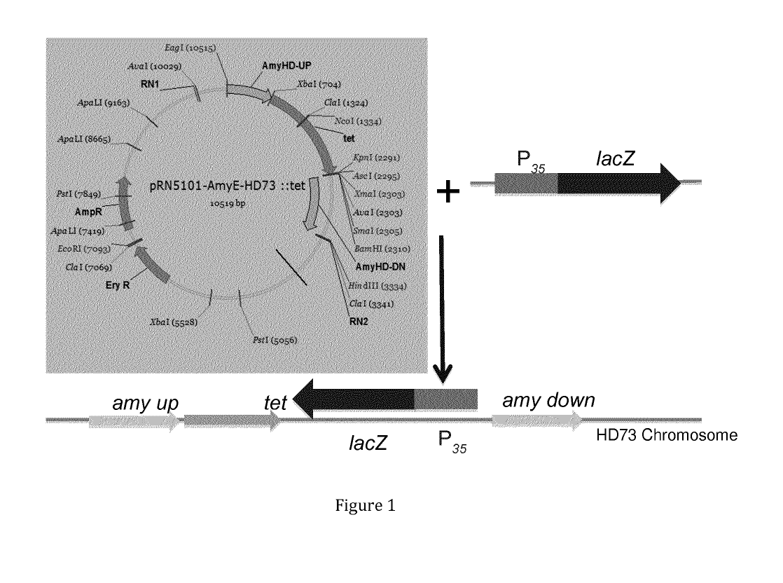

FIG. 1 shows the construction of a Bacillus thuringiensis strain.

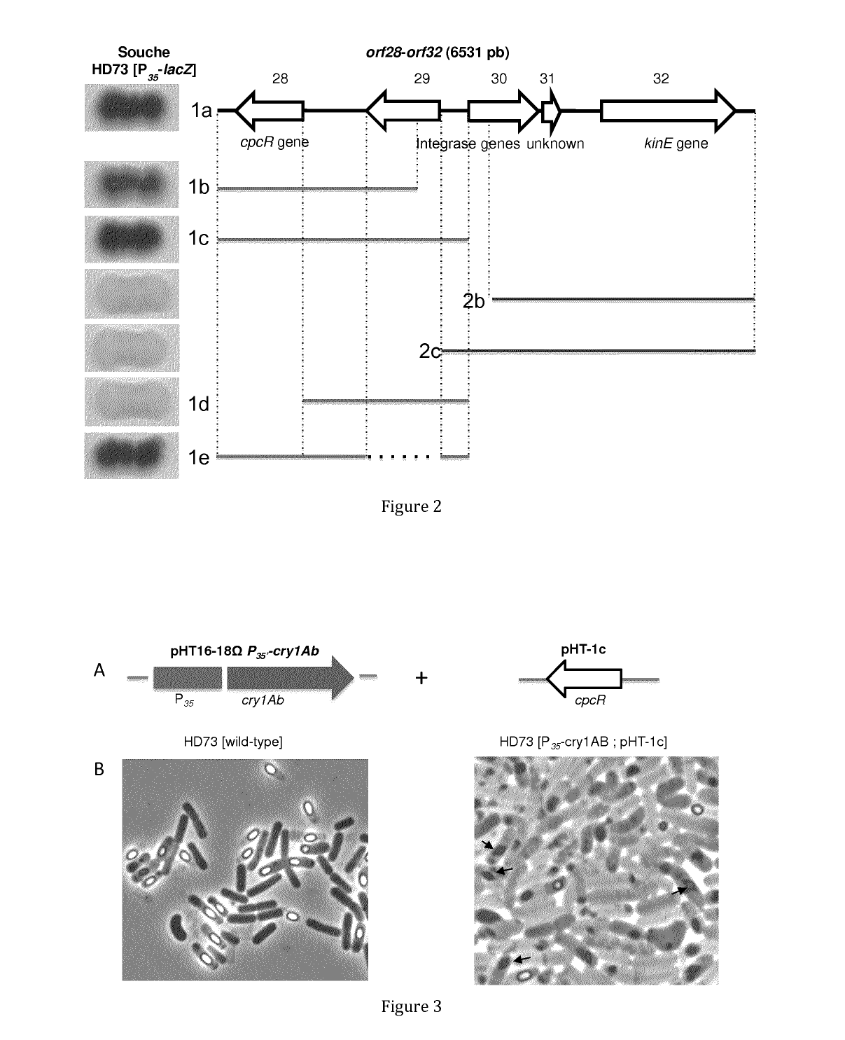

FIG. 2 shows the characterization of the LM1212 DNA fragment carrying the cpcR gene activating transcription from the P₃₅ promoter.

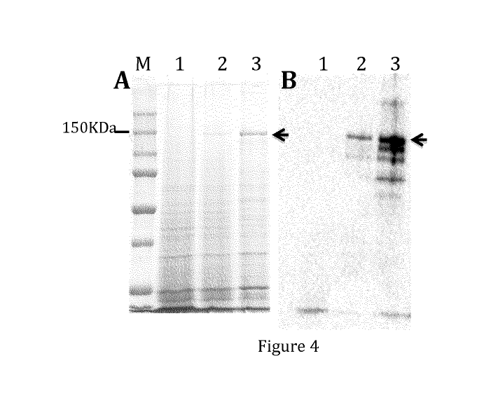

FIG. 3 shows the expression of cry1Ab under the control of CpcR and P₃₅ in a kurstaki strain. A) Plasmids pHT16-18ΩP35′-cry1Ab and pHT-1c. B) Phase-contrast microscopy of the strain kurstaki HD73 transformed with plasmids pHT16-18ΩP35′-cry1Ab and pHT-1c. The arrows indicate some crystal inclusions.

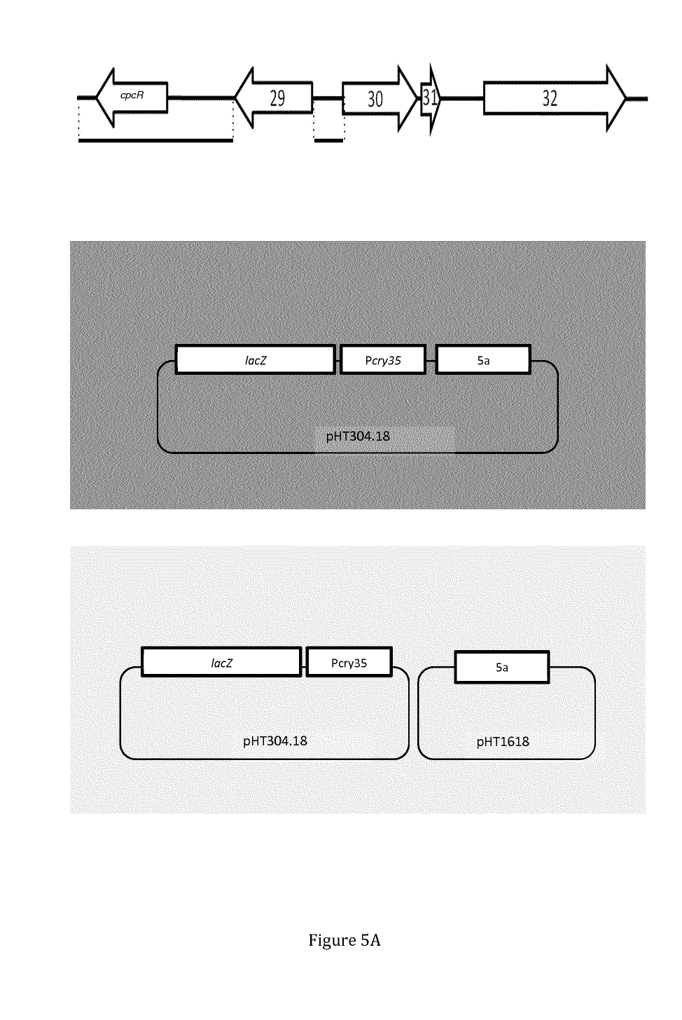

FIG. 4 shows the analysis of a Cry1 Ab toxin produced in a kurstaki strain under the control of CpcR and P₃₅. A) SDS-Page. M corresponds to molecular weight markers. 1. 20 μL of crude extract from colonies of the strain kurstaki HD73 (pHT16-18, pHT304). 2. 20 μL of crude extract from colonies of the strain kurstaki HD73 (pHT16-18ΩP35′-cry1Ab, pHT304). 3. 20 μL of crude extract from colonies of the strain kurstaki HD73 (pHT16-18ΩP35′-cry1Ab, pHT-1c). The crude extracts were prepared in the same way and the use of 20 μL of crude extract means that the same amount of each preparation is loaded on the SDS-Page. B) Western blotting of lanes 1, 2 and 3 with antisera against Cry1Ab.

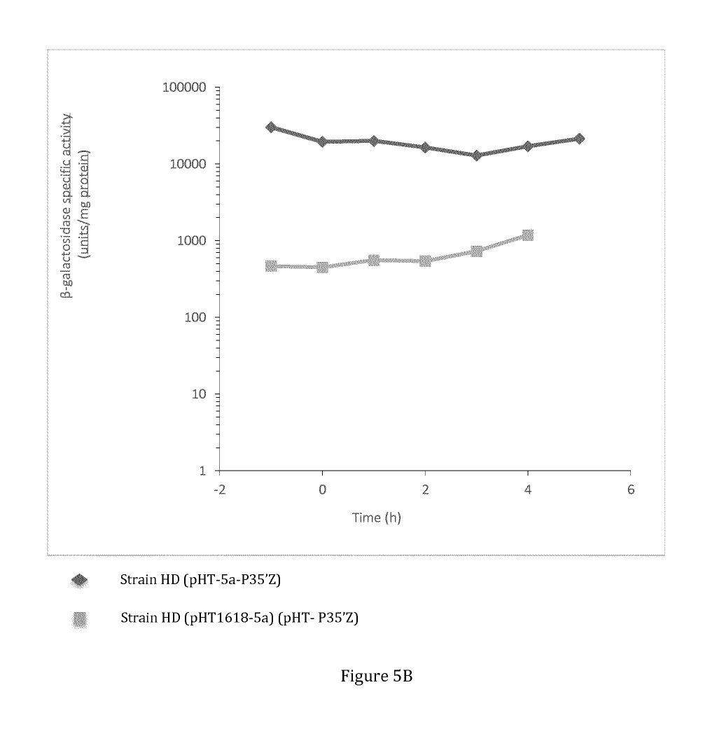

FIG. 5 shows the transcriptional analysis of the P35′lacZ fusion. A. Detail of the constructs. The upper panel shows the DNA fragment 5a carrying the cpcR gene.

The two lines below the schematic representation of the cpcR locus indicates the DNA regions used to create fragment 5a. The middle panel highlighted in dark grey shows the organization of plasmid (pHT-5a-P35′Z) and the lower panel highlighted in light grey shows the organization of plasmids (pHT1618-5a) and (pHT-P35′Z). B. β-galactosidase assay of strains HD (pHT-5a-P35′Z) in dark grey lozenges and HD (pHT1618-5a) (pHT-P35′Z) in light grey squares. The time indicated is relative to t0 which indicates the beginning of the transition between exponential and stationary phase.

### Example 1: Construction of a Bacillus Thuringiensis Strain to Screen for the Regulator of the Expression from the P₃₅ Promoter

The previous results obtained by the inventors suggested that, in the LM1212 strain, a regulation system limits the production of toxins to the subpopulation of non-sporulating cells. Thus, to screen the LM1212 genes responsible for the regulation of the P₃₅ promoter, the promoter directing the expression of the cry gene, the inventors introduced a reporting system into a Bacillus thuringiensis strain, as described thereafter.

### 1) Materials and Methods

The Bt strain kurstaki HD73 Cry⁻ is chosen to clone the LM1212 genes responsible for the activation of the P₃₅ promoter and for the negative effect on sporulation.

The thermosensitive plasmid pRN5101-AmyE-HD73::tet is first constructed following the same procedure as described in Verplaetse et al., 2015, except that the antibiotic resistance cassette confers resistance to tetracycline instead of spectinomycin and that the amyE flanking regions used for homologous recombination were amplified using Bt HD73 genomic DNA instead of Bt 407. A P_{35′}-lacZ transcriptional fusion is then cloned between the amy up and amy down fragments cloned from the kurstaki strain. The P_{35′}-lacZ fusion is then introduced at the amyE locus of the kurstaki HD73 chromosome by homologous recombination (FIG. 1).

### 2) Results

The resulting strain is designated HD73 Cry⁻ [amyE::P_{35′}-lacZ].

### Example 2: Characterization of the LM1212 Genes Activating Transcription from the P₃₅ Promoter

i. Localization of the Genes Responsible for the Expression from the P₃₅ Promoter on the DNA of LM1212 Strain

### 1) Materials and Methods

The DNA fragment ORF28-ORF32 from plasmid PLM248 of strain LM1212 is cloned into plasmid pHT304 (Arantes, O. et al., 1991) and the resulting plasmid is called pHT-1a. pHT-1a is then introduced into the Bt strain HD73 Cry⁻ [amyE::P_{35′}-lacZ] and the recombinant clones are isolated on HCT plates (0.7% casein hydrolysate, 0.5% tryptone, 0.68% KH2 PO4, 0.012% MgSO4_7H2O, 0.00022% MnSO4_4H2O, 0.0014% ZnSO4_7H2O, 0.008% ferric ammonium citrate, 0.018% CaCl₂_4H2O, 0.3% glucose, pH 7.2) (Lecadet et al., 1980; Verplaetse et al., 2016) containing yeast extract (0.05%), glucose 0.3% and X-Gal (100 μg/mL).

### 2) Results

The parental strain gives white colonies. In sharp contrast, the strain harboring plasmid pHT-1a gives blue colonies (FIG. 2). This result indicates that the genes responsible for the expression from the P₃₅ promoter are located on the DNA fragment 1a corresponding to LM1212 ORF28 to 32.

ii. Identification of the Genes Involved in the Transcriptional Activity

### 1) Materials and Methods

The DNA region ORF28 to 32 is sub-cloned into the pHT304 plasmid and the resulting plasmids are transformed into the Bt strain HD73 Cry⁻ [amyE::P_{35′}-lacZ](FIG. 2).

### 2) Results

It appears that the ORF28 encoding a transcriptional regulator is sufficient to activate P₃₅-directed transcription. However, this activity is obtained if a short DNA fragment located just upstream of the ORF29 is fused upstream from ORF28 (Fragment 1e, FIG. 2). ORF28 encodes a transcriptional regulator belonging to the family of response regulators, suggesting that it should act as a two-component system in association with a histidine kinase. Such a kinase may be encoded by ORF32.

However, results show that ORF28 alone is able to activate the P₃₅ promoter. In silico analysis of these genes (ORF28 and 32) indicates that ORF28 is unique and is not found in the available sequences of the bacteria of the B. cereus group (about 100 genomes).

In contrast, ORF32 is highly conserved among all the bacteria of the B. cereus group, including the LM1212.

Thus, ORF28 may function as a response regulator activated by the histidine kinase naturally present in the kurstaki HD73 strain.

iii. Effect of the Plasmid pHT-1c on the Sporulation of the Bacteria

### 1) Materials and Methods

pHT-1c, which corresponds to the plasmid pHT304 carrying the gene ORF28 and a promoter region, is introduced in Bt strain HD73 Cry⁻ [amyE::P_{35′}-lacZ].

### 2) Results

The introduction of pHT-1c in Bt strain HD73 Cry⁻ [amyE::P_{35′}-lacZ] negatively affects sporulation by a 10-fold factor (Table 1).

TABLE 1
Effect of the DNA fragment 1c carrying the cpcR gene on
the sporulation of B. thuringiensis stain HD73. The
results are the mean of two independent experiments.

| | Heat-resistant |
|---|---|
| Strains | spores (CFU/ml)^{a} |
| HD73 amyE::Pcry35-lacZ (pHT304) | 5.25E+08 (±5.25E+07) |
| HD73 amyE::Pcry35-lacZ (pHT304-1c) | 4.99E+07 (±9.42E+06) |

^{a}The bacteria were grown in liquid HCT medium (containing Yeast Extract 0.05% and Glucose 0.3%) at 30° C. 48 h after inoculation, aliquots were heated at 80° C. for 12 mins. The cells were plated and CFUs corresponding to heat-resistant spores were then counted.

iv. Conclusion

These data indicate that the gene corresponding to ORF28 encodes the regulator (designated as CpcR) responsible for two functions: i) the activation of cry gene expression in the LM1212 strain, and ii) the reduction of the sporulation rate.

The results also suggest that ORF28 functions as a two-component system associated with a kinase existing in all the Bt strains. This gene is responsible for the specific phenotype of the LM1212 strain, which is to differentiate into crystal-producers or spore-formers.

### Example 3: Use of the CpcR Regulator to Produce Cry1Ab in Non-Sporulating Bt Cells

In order to validate the use of CpcR in a Bt strain for reducing its sporulation while maintaining toxin production, the inventors produced recombinant strains of Bt and analyzed the sporulation rate of this recombinant strain and the production of the toxin Cry1Ab.

### 1) Materials and Methods

a) The cry1Ab gene, a typical sporulation-dependent cry gene is PCR amplified from strain kurstaki HD1 (the biopesticide kurstaki HD1 Dipel®) and is cloned downstream from the P₃₅ promoter into plasmid pHT16-18 (Lereclus et al., 1992). The resulting plasmid, pHT16-18Ω P35′-cry1Ab (i.e. the plasmid pHT16-18 carrying the cry1Ab gene from the kurstaki HD1 Dipel® strain under the control of the P₃₅ promoter) is transformed into Bt strain HD73 Cry⁻. The resulting strain was then transformed with the plasmid pHT-1c carrying the cpcR gene. The bacteria were plated on HCT agar plates for 48 h at 30° C. and examined in phase-contrast microscopy (FIG. 3).

b) The proteins produced by these recombinant strains are analyzed by western blotting with antisera against the Cry1Ab toxin (FIG. 4).

### 2) Results

a) The results show the production of typical crystal inclusion in non-sporulating cells of the kurstaki strain harboring plasmids pHT16-18ΩP_{35′}-cry1Ab and pHT-1c. Moreover, the sporulation rate of the recombinant strain carrying pHT16-18ΩP₃₅′-cry1Ab and pHT-1c is significantly lower than that of the wild-type strain.

b) The production of Cry1Ab is strongly increased in the kurstaki strain harboring the cpcR gene and the P₃₅′-cry1Ab fusion (Panels A and B, Lanes 3).

The faint band of Cry1Ab observed in lanes 2 results from a low CpcR-independent expression of cry1Ab.

These results show that the invention provides a new range of biopesticides by the way of production of insecticidal Cry toxins in non-sporulating Bt strains.

### Example 4: Transcription of the P₃₅ Promoter is Higher when Cloned on the Same Plasmid as CpcR

### 1) Materials and Methods

The DNA fragment designated 5a and comprised of ORF28, the intergenic region between ORF28 and ORF29 and the intergenic region between ORF29 and ORF30, was either cloned upstream from a transcriptional fusion between P₃₅ and the reporter gene lacZ on vector pHT304.18 (which has the same skeleton as vector pHT304, but the cloning site is in the opposite orientation to that of vector pHT304), giving plasmid pHT-5a-P35′Z, or alone on vector pHT1618, giving plasmid pHT1618-5a. The latter was transformed in Bt strain HD73 Cry⁻ along with pHT-P35′Z, giving strain HD (pHT1618-5a) (pHT-P35′Z). Plasmid pHT-5a-P35′Z was also transformed in Bt strain HD73 Cry⁻, giving strain HD (pHT-5a-P35′Z). The cells were grown in liquid HCT containing 0.05% yeast extract and 0.3% glucose. Samples were harvested and assayed for β-galactosidase activity (Perchat et al., 2011).

### 2) Results

FIG. 5 shows that even though P35 is active in both strain HD (pHT1618-5a) (pHT-P35′Z) (light grey squares) and HD (pHT-5a-P35′Z) (dark grey lozenges), its activity is higher in strain HD (pHT-5a-P35′Z), in particular it is 14-fold higher after 4 hours (1200 units versus 17000 units, respectively).

### REFERENCES

Agaisse, H. et al., J. Bacteriol. 1995. 177: 6027-6032.
Arantes, O. et al., Gene 1991. 108: 115-119.
Bone, E J. et al., FEMS Microbiol Lett. 1989. 49(2-3):171-177.
Bravo A. et al., Insect Biochem Mol Biol. 2011.41: 423-431.
Crickmore, N. et al., Microbiol. Mol. Biol. Rev. 1998. 62: 807-813.
Deng, C. et al., Toxins 2014. 6: 2194-2209.
Deng C. et al., The ISME Journal. 2015. 9: 286-296.
Fromm et al., Nature 1990. 319:791-793.
Koehler T M, et al. J Bacteriol. 1994. 176(3):586-595.
Lecadet M et al., Microbiology. 1980. 121: 1: 203-212.
Lereclus, D. et al., FEMS Microbiol. Lett. 1989. 60: 211-217.
Lereclus, D. et al., Nature Biotechnology 1992. 10: 418-421.
Lereclus, D. et al., Mol. Microbiol. 1992. 7: 35-46.
Mahillon, J. et al., Eynard & Teissie (Eds.) Springer Lab. Manual 1999. 242-252.
Peng, D. H., et al. J. Appl. Microbiol. 2009. 106:1849-1858
Perchat S. et al., Molecular microbiology. 2011. 82: 619-633
Sanahuja G. et al., Plant Biotechnol Journal. 2011. 9:283-300.
Schnepf, E. et al., Microbiol. Mol. Biol. Rev. 1998. 62: 775-806.
Trieu-Cuot, P. et al., FEMS Microbiol Lett 1987. 48: 289-294.
Van Frankenhuizen, K. et al., J. Invertebr. Pathol. 2009. 101: 1-16.
Verplaetse, E. et al., MBio 2015. 6: e136008-15.
Verplaetse E. et al., Res Microbiol. 2016. doi: 10.1016/j.resmic.2016.03.006.
Wei J-Z. et al., PNAS 2003. 100(5):2760-2765.

1. A method of reducing sporulation of a recombinant strain of Bacillus thuringiensis, comprising the expression of a cpcR regulator gene of the sequence SEQ ID NO: 1 in said Bacillus thuringiensis.

2. The method of claim 1, wherein said CpcR regulator directs the expression of promoters of genes encoding Cry and Cyt proteins, said promoters having the sequence SEQ ID NO: 2 as follows:
X₁TGAAX₂AAAAX₃X₄X₅X₆CAX₇X₈AX₉ATTTX₁₀CX₁₁TCX₁₂X₁₃X₁₄X₁₅X₁₆T
X₁₇X₁₈AX₁₉ATGTX₂₀X₂₁TX₂₂GX₂₃TAX₂₄AX₂₅TX₂₆X₂₇X₂₈X₂₉AX₃₀X₃₁TX₃₂
X₃₃, with:
X₁ = A or G; X₂ = C or T; X₃ = A or T; X₄ = A, T or
G; X₅ = A, C or T; X₆ = A or G; X₇ = C or T; X₈ = A
or C X₉ = A, T or G; X₁₀ = A or C; X₁₁ = A or C;
X₁₂ = A, C or T; X₁₃ = A, G or T; X₁₄ = A or C; X₁₅ =
A, G or T; X₁₆ = A or G; X₁₇ = A or T; X₁₈ = A, C or
T; X₁₉ = C or T; X₂₀ = A or C; X₂₁ = A, C or G; X₂₂ =
A or T; X₂₃ = C or T; X₂₄ = G or T; X₂₅ = C or T;
X₂₆ = G or T; X₂₇ = A or G; X₂₈ = A or T; X₂₉ = A, G
or T; X₃₀ = C, G or T; X₃₁ = A or G; X₃₂ = A or G;
X₃₃ = C or T.

3. The method of claim 2, wherein the promoter is selected from the group consisting of: P₃₂ of the sequence SEQ ID NO: 3, P₄₁ of the sequence SEQ ID NO: 4, P₃₅ of the sequence SEQ ID NO: 5, P₄₅ of the sequence SEQ ID NO: 6.

4. The method of claim 1, wherein the genes encoding toxins are cry genes or cyt genes.

5. The method of claim 1, wherein the strain of Bacillus comprises cry1, cry2, cry3, cry4, cry5, cry6, cry8, cry9, cry11, cry14, cry21, cyt1 or cyt2 genes.

6. A recombinant strain of Bacillus thuringiensis comprising:
at least one gene encoding Cry and/or Cyt toxins,
at least one promoter having the sequence SEQ ID NO: 2, allowing the expression of said at least one gene encoding Cry and/or Cyt toxins, and
a cpcR regulator gene of sequence SEQ ID NO: 1, which directs the expression of said at least one promoter.

7. The recombinant strain of Bacillus thuringiensis as claimed in claim 6, wherein the promoter is selected from the group consisting of: P₃₂ of the sequence SEQ ID NO: 3, P₄₁ of the sequence SEQ ID NO: 4, P₃₅ of the sequence SEQ ID NO: 5, P₄₅ of the sequence SEQ ID NO: 6.

8. The recombinant strain of Bacillus thuringiensis as claimed in claim 6, wherein the promoter is cloned on the same plasmid as the cpcR regulator gene.

9. The recombinant strain of Bacillus thuringiensis as claimed in claim 6, wherein the genes encoding toxins are cry genes or cyt genes.

10. The recombinant strain of Bacillus thuringiensis as claimed in claim 6, wherein the strain of Bacillus comprises cry1, cry2, cry3, cry4, cry5, cry6, cry8, cry9, cry11, cry14, cry21, cyt1 or cyt2 genes.

11. Use of a recombinant strain of Bacillus thuringiensis as claimed in claim 6 as biopesticide.

12. The use of the recombinant strain of Bacillus thuringiensis as claimed in claim 11 for protecting cultures.

13. The use of the recombinant strain of Bacillus thuringiensis as claimed in claim 11 to control vectors.

14. The use of the recombinant strain of Bacillus thuringiensis as claimed in claim 10 to control nematodes.

15. A method for obtaining a recombinant strain of Bacillus thuringiensis, comprising the steps of introducing in said strain both:
(1) a genetic construction comprising at least one gene encoding a toxin under the control of a promoter having the sequence SEQ ID NO: 2, and
(2) an expression system comprising the CpcR regulator of the sequence SEQ ID NO: 1.

16. The method for obtaining a recombinant strain of Bacillus thuringiensis, as claimed in claim 15, wherein the genetic construction as defined in step 1 of claim 15 and the expression system as defined in step 2 of claim 15 are on the same plasmid.

17. The use of the recombinant strain of Bacillus thuringiensis as claimed in claim 13, wherein the vectors transmit pathogens responsible for mammalian diseases.

18. The use of the recombinant strain of Bacillus thuringiensis as claimed in claim 17, wherein the vectors are mosquitos.

19. The use of the recombinant strain of Bacillus thuringiensis as claimed in claim 14, wherein the nematodes cause mammalian disease.
